(19) United States
(12) Patent Application Publication
(10) Pub. No.: US 2024/0150786 A1
(43) Pub. Date: May 9, 2024

(54) DOUBLE AND SINGLE MUTATED PLANT HAVING INCREASED DEFENSE RESPONSE AND REDUCED DISEASE LEVELS, AND METHODS TO GENERATE SAME

(71) Applicants: THE STATE OF ISRAEL, MINISTRY OF AGRICULTURE & RURAL DEVELOPMENT, AGRICULTURAL RESEARCH ORGANIZATION, Rishon Lezion (IL); RAMOT AT TEL AVIV UNIVERSITY LTD., Tel-Aviv (IL)

(72) Inventors: Maya BAR, Ramat Gan (IL); Meirav LEIBMAN, Tel Aviv (IL); Adi AVNI, Tel-Aviv (IL)

(21) Appl. No.: 18/281,304

(22) PCT Filed: Mar. 10, 2022
(86) PCT No.: PCT/IL2022/050277

§ 371 Date: Sep. 10, 2023

Related U.S. Application Data
(60) Provisional application No. 63/159,496, filed on Mar. 11, 2021.

(51) Int. Cl. C12N 15/82

(52) U.S. Cl. CPC C12N 15/8282; C12N 15/8281; C12N 15/8286

(57) ABSTRACT

This invention is related to a double mutant plant line that harbors impaired NRC4a (NLR required for cell death) and NRC4b genes. The said double mutant plant has an increased defense response and a reduction in disease levels compared to a non-mutated or a single mutation mutated plant of the same population. This invention is also related to the method for increasing a plant defense response and for reducing plant diseases levels, comprised by (a) configuring one or more nucleic acid sequences to target and impair at least two plant NRC (NLR required for cell death) or NRC ortholog genes, (b) applying a gene editing process utilizing said one or more nucleic acid sequences, (c) obtaining at least one double mutant plant line, harboring two impaired NRC (NLR required for cell death) genes, and (d) growing said plant.

### FIELD OF INVENTION

The present invention relates to the field of agriculture, plant's pathogens resistance, and more particularly the present invention concerns crisper/cas single and double mutated plant lines with increased resistance and response to pathogen infection.

### BACKGROUND OF INVENTION

Crop pathogens reduce the yield and quality of agricultural production. They cause substantial economic losses and reduce food security at household, national and global levels (Savary et. al, Nat Ecol Evol 2019 March; 3(3):430-439). Herbicides and pesticides have been used to control, eliminate or destroy pests in order to protect crops. Unfortunately, herbicides and pesticides have harmful effects directly or indirectly on soil, environment, surface and ground water natural flora and fauna, aquatic life which ultimately adversely influence the human beings and livestock. The impact of herbicides and pesticides on atmosphere and community health is of great significance regardless of their noticeable benefits and have led to heavy regulation and to a worldwide ban of many herbicides and pesticides (Rashid B. et al, https://link.springer.com/book/10.1007/978-90-481-9370-7). Plants are constantly challenged by potential pathogens. To defend themselves, plants utilize an innate two-tiered immune system: pattern triggered immunity (PTI) and effector triggered immunity (ETI) (Kachroo et al., 2017). The first line is formed by pattern recognition receptors (PRRs), located at the cell surface, that recognize microbe-associated molecular patterns (MAMPs), leading to PTI (Boutrot and Zipfel, 2017). Upon MAMP recognition, PRRs activate a signaling cascade, that leads to robust transcriptional changes and physiological changes in order to restrict pathogen attack (Bigeard et al., 2015). R (recognition) proteins can be classified based on their structure to kinase proteins and nucleotide binding (NB) leucine-rich repeat (LRR) proteins (NLR) (Monteiro and Nishimura, 2018). NLRs typically consist of a highly polymorphic C-terminal LRR domain that is thought to confer recognition specificity, and a central NB-ARC domain that is thought to function as a molecular switch (Monteiro and Nishimura, 2018). NLRs can be further classified into two subgroups, based on their N-terminal domain: NLRs containing toll-interleukin 1 receptor (TIR) domain (TNLs) or coiled coil (CC) domain (CNLs) (Monteiro and Nishimura, 2018). Several regulatory mechanisms of NLR activity have been demonstrated, including intramolecular regulation and homo- and heterodimerization (Jubic et al., 2019; Wu et al., 2017). In Solanaceae, a subfamily of NLRs termed NLR required for cell death (NRC) emerges as a key family of NLRs (Wu et al., 2017). The tomato NLR-NRC4a functions as an h-NLR and is required for defense signaling mediated by the xylanase receptor LeEIX2 (Leibman-Markus et al., 2018). NRC4a CRISPRed plants, encoding a 67 aa truncated protein variant, resulted in a gain of function mutant that displayed intensified defense responses, and presented a higher resistance to B. cinerea (Leibman-Markus et al., 2018b). Further the NRC4a crispr mutant possess a broad-spectrum disease resistance in tomato.

With plants being constantly exposed to evolving pathogens, crop losses pose a major threat to global food security, combined with the understanding of the plant immune system and in view of the above there is still a long felt need for the development of disease resistant plants and methods to increased immune responses in plants.

### SUMMARY OF THE INVENTION

It is thus an object of the present invention to disclose_a double mutant plant line characterized by an increased defense response compared to a non-mutated or a single mutation mutated plant of the same population, wherein said double mutant plant harbors impaired NRC4a (NLR required for cell death) and NRC4b genes.

It is another object of the present invention to disclose_a double mutant plant line characterized by a reduction in disease levels compared to a non-mutated or a single mutation mutated plant of the same population, wherein said double mutant plant harbors impaired NRC4a (NLR required for cell death) and NRC4b genes.

It is another object of the present invention to disclose_he aforementioned double mutant plant line, wherein said plant is a double mutant homozygous plant to the NRC4a and/or NRC4b impaired gene.

It is another object of the present invention to disclose_the aforementioned double mutant plant line, wherein said plant expresses a truncated NRC4a and/or NRC4b proteins.

It is another object of the present invention to disclose_the aforementioned truncated NRC4a and NRC4b proteins, wherein at least one of said truncated proteins is characterized by at least one increased function compared to the full-length proteins.

It is another object of the present invention to disclose_the aforementioned truncated NRC4a and NRC4b proteins, wherein at least one of said truncated proteins possess at least one enhanced activity of the native proteins.

It is another object of the present invention to disclose_the aforementioned double mutant plant line, wherein said plant is a member of the Solanaceae family.

It is another object of the present invention to disclose_the aforementioned double mutant plant, wherein said plant is selected from a group consisting of Solanum lycopersicum, Nicotiana tabacum, Solanum tuberosum, Solanum melongena, Capsicum annum, Petunia hybrid, a NRC4a and/or NRC4b genes expressing plant, a NRC4a and/or NRC4b ortholog gene expressing plant, and any combination thereof.

It is another object of the present invention to disclose the mutant aforementioned plant line, wherein said double mutant plant is mutated by means of a gene editing system.

It is another object of the present invention to disclose the gene editing system, wherein said gene editing system is selected from a group consisting of: gene editing agent, meganucleases, zinc finger nucleases, TALEN, Crispr/Cas and any combination thereof.

It is another object of the present invention to disclose the aforementioned gene editing system, wherein said gene editing system is a crispr/cas system.

It is another object of the present invention to disclose the aforementioned single mutation mutated plant, wherein said single mutation is located at either the NRC4a or NRC4b genes.

It is another object of the present invention to disclose the aforementioned double mutant plant, wherein said increased defense response is further increased upon interaction with a bio control agent (BCA).

It is another object of the present invention to disclose the aforementioned double mutant plant line, wherein said reduction in disease levels is further increased upon interaction with a bio control agent (BCA).

It is another object of the present invention to disclose the aforementioned bio control agent, wherein said bio control agent is selected from a group consisting of:
a. insects,
b. microorganisms such as fungi, bacteria, oomycets, viruses,
c. chemicals such as nucleic acids, hormones, hormone analogs, natural products, organic compounds.

It is another object of the present invention to disclose a method for increasing a plant defense response, comprising the following steps:
a. configuring one or more nucleic acid sequences to target and impair at least two plant NRC (NLR required for cell death) or NRC ortholog genes,
b. applying a gene editing process utilizing said one or more nucleic acid sequences,
c. obtaining at least one double mutant plant line, harboring two impaired NRC (NLR required for cell death) genes.

It is another object of the present invention to disclose a method for increasing a plant defense response, comprising the following steps:
a. configuring one or more nucleic acid sequences to target and impair at least two plant NRC (NLR required for cell death) or NRC ortholog genes,
b. applying a gene editing process utilizing said one or more nucleic acid sequences,
c. obtaining at least one double mutant plant line, harboring two impaired NRC (NLR required for cell death) genes.
d. growing said plant.

It is another object of the present invention to disclose the aforementioned method, wherein said nucleic acid sequence is selected from a group consisting of DNA, RNA, tRNA, gRNA, cDNA, hybrid nucleic acid, or any combination thereof.

It is another object of the present invention to disclose the aforementioned method, wherein said nucleic acid sequence is gRNA of the CRISPR/Cas gene editing system.

It is another object of the present invention to disclose the aforementioned method wherein said plant is a member of the Solanaceae family.

It is another object of the present invention to disclose the aforementioned method, wherein said plant is selected from a group consisting of, Solanum lycopersicum, Nicotiana tabacum, Solanum tuberosum, Solanum melongena, Capsicum annum, Petunia hybrid, a NRC4a and NRC4b genes expressing plant, a NRC4a and NRC4b ortholog genes expressing plant and any combination thereof.

It is another object of the present invention to disclose the aforementioned method, wherein said NRC (NLR required for cell death) genes are the NRC4a and NRC4b genes.

It is another object of the present invention to disclose the aforementioned double mutant plant line, wherein said plant is a double mutant homozygous to at least one of the NRC4a and NRC4b impaired gene.

It is another object of the present invention to disclose the aforementioned method, wherein said double mutant plant line express at least one truncated NRC4a and/or NRC4b proteins.

It is another object of the present invention to disclose the aforementioned truncated NRC4a and/or NRC4b proteins, wherein said truncated NRC4a and/or NRC4b proteins are characterized by at least one increased function compared to the full-length proteins.

It is another object of the present invention to disclose the aforementioned NRC4a and/or NRC4b truncated proteins, wherein said NRC4a and/or NRC4b truncated proteins possess at least one enhanced activity of the native proteins.

It is another object of the present invention to disclose a mutated plant line characterized by an increased defense response compared to a non-mutated plant of the same population, wherein said mutated plant harbors an impaired NRC4b (NLR required for cell death) gene.

It is another object of the present invention to disclose a mutated plant line characterized by a reduction in disease levels compared to a non-mutated plant of the same population, wherein said mutated plant harbors an impaired NRC4b (NLR required for cell death) gene.

It is another object of the present invention to disclose the aforementioned mutated plant line, wherein said mutated plant line is homozygous to the impaired NRC4b gene.

It is another object of the present invention to disclose the aforementioned mutated plant line, wherein said mutated plant line expresses a truncated NRC4b protein.

It is another object of the present invention to disclose the aforementioned truncated NRC4b protein, wherein said truncated NRC4b protein is characterized by at least one increased function compared to the full-length protein.

It is another object of the present invention to disclose the aforementioned truncated NRC4b protein, wherein said NRC4b protein possesses at least one enhanced activity of the native protein.

It is another object of the present invention to disclose the aforementioned mutated plant line, wherein said mutated plant line is a member of the Solanaceae family.

It is another object of the present invention to disclose the aforementioned mutated plant line, wherein said plant is selected from a group consisting of Solanum lycopersicum, Nicotiana tabacum, Solanum tuberosum, Solanum melongena, Capsicum annum, Petunia hybrida, a NRC4b gene expressing plant, a plant expressing NRC4b functional ortholog, and any combination thereof.

It is another object of the present invention to disclose the aforementioned mutated plant line, wherein said increased defense response is further increased upon interaction with a bio control agent (BCA).

It is another object of the present invention to disclose the aforementioned mutated plant line, wherein said reduction in disease levels is further increased upon interaction with a bio control agent (BCA).

It is another object of the present invention to disclose the aforementioned bio control agent, wherein said bio control agent is selected from a group selected from a group consisting of:
a. insects,
b. microorganisms such as fungi, bacteria, oomycets, viruses,
c. chemicals such as nucleic acids, hormones, hormone analogs, natural products, organic compounds.

It is another object of the present invention to disclose the aforementioned mutated plant line, wherein said mutated plant line is mutated by means of a gene editing system.

It is another object of the present invention to disclose the aforementioned gene editing system, wherein said gene editing system is selected from a group consisting of: gene editing agent, meganucleases, zinc finger nucleases, TALEN, Crispr/Cas and any combination thereof.

It is another object of the present invention to disclose the aforementioned gene editing system, wherein said gene editing system is a crispr/cas 9 system.

It is another object of the present invention to disclose a method for reducing a plant disease levels, comprising the following steps:
a. configuring at least one nucleic acid sequences to target and impair a plant NRC4b (NLR required for cell death) or NRC4b ortholog genes,
b. applying a gene editing process utilizing said at least one nucleic acid sequence,
c. obtaining at least one mutant plant line, harboring an impaired NRC4b (NLR required for cell death) gene.

It is another object of the present invention to disclose a method for reducing a plant disease levels, comprising the following steps:
a. configuring at least one nucleic acid sequences to target and impair a plant NRC4b (NLR required for cell death) or NRC4b ortholog genes,
b. applying a gene editing process utilizing said at least one nucleic acid sequences,
c. obtaining at least one mutant plant line, harboring an impaired NRC4b (NLR required for cell death) genes.
d. growing said plant.

It is another object of the present invention to disclose the aforementioned method, wherein said nucleic acid sequence is selected from a group consisting of DNA, RNA, tRNA, gRNA, cDNA, hybrid nucleic acid, or any combination thereof.

It is another object of the present invention to disclose the aforementioned method, wherein said nucleic acid sequence is gRNA of the CRISPR/Cas gene editing system.

It is another object of the present invention to disclose the aforementioned method, wherein said plant is a member of the Solanaceae family.

It is another object of the present invention to disclose the aforementioned method, wherein said plant is selected from a group consisting of, Solanum lycopersicum, Nicotiana tabacum, Solanum tuberosum, Solanum melongena, Capsicum annum, Petunia hybrida, a NRC4b genes expressing plant, a NRC4b ortholog genes expressing plant and any combination thereof.

It is another object of the present invention to disclose the aforementioned method according to any one gene is the NRC4b gene.

It is another object of the present invention to disclose the aforementioned mutant plant, wherein said mutant plant line is a mutant homozygous to the NRC4b impaired gene.

It is another object of the present invention to disclose the aforementioned method, wherein said mutant plant line express a truncated NRC4b protein.

It is another object of the present invention to disclose the aforementioned truncated NRC4b protein, wherein said truncated NRC4b protein is characterized by at least one increased function compared to the full-length protein.

It is another object of the present invention to disclose the aforementioned truncated NRC4b protein, wherein said NRC4b protein possesses at least one enhanced activity of the native protein.

It is another object of the present invention to disclose a method for increasing a plant defense response, comprising the following steps:
a. configuring at least one nucleic acid sequences to target and impair a plant NRC4b (NLR required for cell death) or NRC4b ortholog genes,
b. applying a gene editing process utilizing said at least one nucleic acid sequence,
c. obtaining at least one mutant plant line, harboring an impaired NRC4b (NLR required for cell death) gene.

It is another object of the present invention to disclose a method for increasing a plant defense response, comprising the following steps:
a. configuring at least one nucleic acid sequences to target and impair a plant NRC4b (NLR required for cell death) or NRC4b ortholog genes,
b. applying a gene editing process utilizing said at least one nucleic acid sequences,
c. obtaining at least one mutant plant line, harboring an impaired NRC4b (NLR required for cell death) gene.
d. growing said plant.

It is another object of the present invention to disclose the aforementioned method, wherein said nucleic acid sequence is selected from a group consisting of DNA, RNA, tRNA, gRNA, cDNA, hybrid nucleic acid, or any combination thereof.

It is another object of the present invention to disclose the aforementioned method, wherein said nucleic acid sequence is gRNA of the CRISPR/Cas gene editing system.

It is another object of the present invention to disclose the aforementioned method, wherein said plant wherein said plant is a member of the Solanaceae family.

It is another object of the present invention to disclose the aforementioned method, wherein said plant is selected from a group consisting of, Solanum lycopersicum, Nicotiana tabacum, Solanum tuberosum, Solanum melongena, Capsicum annum, Petunia hybrida a NRC4b genes expressing plant, a NRC4b ortholog genes expressing plant and any combination thereof.

It is another object of the present invention to disclose the aforementioned method, wherein said NRC (NLR required for cell death) gene is the NRC4b gene.

It is another object of the present invention to disclose the aforementioned method, wherein said mutant plant line expresses a truncated NRC4b protein.

It is another object of the present invention to disclose the aforementioned truncated NRC4b protein, wherein said truncated NRC4b protein is characterized by at least one increased function compared to the full-length protein.

It is another object of the present invention to disclose the aforementioned truncated NRC4 protein, wherein said NRC4b protein possesses at least one enhanced activity of the native protein.

It is another object of the present invention to disclose a method for reducing plant disease levels, comprising the following steps:
a. configuring one or more nucleic acid sequences to target and impair at least two plant NRC (NLR required for cell death) or NRC ortholog genes,
b. applying a gene editing process utilizing said one or more nucleic acid sequences,
c. obtaining at least one double mutant plant line, harboring two impaired NRC (NLR required for cell death) genes.

It is another object of the present invention to disclose a method for reducing a plant disease levels, comprising the following steps:
a. configuring one or more nucleic acid sequences to target and impair at least two plant NRC (NLR required for cell death) or NRC ortholog genes,
b. applying a gene editing process utilizing said one or more nucleic acid sequences,
c. obtaining at least one double mutant plant line, harboring two impaired NRC (NLR required for cell death).
d. growing said plant.

It is another object of the present invention to disclose the aforementioned method according to, wherein said nucleic acid sequence is selected from a group consisting of DNA, RNA, tRNA, gRNA, cDNA, hybrid nucleic acid, or any combination thereof.

It is another object of the present invention to disclose the aforementioned method, wherein said nucleic acid sequence is gRNA of the CRISPR/Cas gene editing system.

It is another object of the present invention to disclose the aforementioned method, wherein said plant wherein said plant is a member of the Solanaceae family.

It is another object of the present invention to disclose the aforementioned method, wherein said plant is selected from a group consisting of: Solanum lycopersicum, Nicotiana tabacum, Solanum tuberosum, Solanum melongena, Capsicum annum, Petunia hybrid, a NRC4a and NRC4b genes expressing plant, a NRC4a and NRC4b ortholog genes expressing plant and any combination thereof.

It is another object of the present invention to disclose the aforementioned method, wherein said NRC (NLR required for cell death) genes are the NRC4a and NRC4b genes.

It is another object of the present invention to disclose the aforementioned method, wherein said double mutant plant line is a double mutant homozygous to the NRC4a and NRC4b impaired genes.

It is another object of the present invention to disclose the aforementioned method, wherein said double mutant plant line express truncated NRC4a and NRC4b proteins.

It is another object of the present invention to disclose the aforementioned truncated NRC4a and NRC4b proteins, wherein said NRC4a and NRC4b truncated proteins are characterized by at least one increased function compared to the full-length proteins.

It is another object of the present invention to disclose the aforementioned truncated NRC4a and NRC4b proteins, wherein said NRC4a and NRC4b proteins possess at least one enhanced activity of the native proteins.

These exemplary embodiments are mentioned not to limit or define the invention, but to provide examples of embodiments of the invention to aid understanding thereof.

Exemplary embodiments are discussed in the Detailed Description, and further description of the invention is provided there. Advantages offered by the various embodiments of the present invention may be further understood by examining this specification.

### BRIEF DESCRIPTION OF THE FIGURES

The accompanying drawings, which are included to provide a further understanding of the invention and are incorporated in and constitute a part of this specification, illustrate embodiments of the invention and together with the description serve to explain the principles of the invention.

These and other features, aspects, and advantages of the present invention are better understood when the following Detailed Description is read with reference to the accompanying drawings, wherein:

FIG. 1 depicting a graphic presentation of reactive oxygen spices (ROS) generation as a result of over expression of the NRC4a and NRC4b mutated genes.

FIG. 2 depicting a schematic presentation of the DNA and amino acid sequences of several mutants of the present invention.

FIG. 3 depicting a schematic presentation of the DNA and amino acid sequences of a double mutant of the present invention.

FIG. 4 depicting a graphic presentation of the lesion area (mm) in different plant lines.

FIG. 5 depicting a graphic presentation of ethylene production of the present invention.

FIG. 6a-6h depicting a graphic presentation of the increase resistance of the NRC4 mutants to various pathogens.

FIG. 7 depicting a graphic representation of the phenotypic parameters of the NRC4 mutants compared to WT.

### DETAILED DESCRIPTION OF PREFERRED EMBODIMENTS

The following description is provided, alongside all chapters of the present invention, so as to enable any person skilled in the art to make use of the invention and sets forth the best modes contemplated by the inventor of carrying out this invention. Various modifications, however, are adapted to remain apparent to those skilled in the art, since the generic principles of the present invention have been defined specifically to provide

The term “gene editing” refers hereinafter to a type of genetic engineering in which DNA undergoes modifications, insertions, deletions or replacement in the genome of a living organism in a site-specific manner.

The term “CRISPER/Cas” refers hereinafter to a gene editing technique in, it enables gene editing in vivo with extremely high precision. It is. Based on a synthetic guide RNA (gRNA) delivering the Cas nuclease to a specific desired location in the cell's genome, allowing deletions or insertion of existing genes in vivo.

The term “gRNA/guide RNA” refers hereinafter to a synthetic RNA sequence, which is an integral component of the gene editing CRISPR/Cas system. It is a non-coding short RNA sequences that first binds to the Cas endonuclease and guides it to a specific location in the cell's genome, then binds to a complementary target DNA sequences where the Cas cleaves the target DNA strand.

The term “homozygous line” refers hereinafter to a homozygous plant which maintains a high degree of consistency for particular characters determined by the gene throughout the subsequent generations, true to type progenies-pure lines (Nishat et al. J Biol Methods. 2016; 3(3)).

The term “plant” refers hereinafter to any of a plant product (roots, stem, foliage, fruits, bulbs) that can be cultivated and harvested extensively to be used as a commodity or for sustainability.

The term “defense response” or “plant defense response” refers hereinafter to an activity of the plant's immune system, which can be triggered by microbial molecules.

In some cases, plant pattern recognition receptors (PRRs) sense or detect different types of microbial molecules, generating a response by activation of a defense mechanism.

Typical manifestations of plant defense include, but are not limited to: Ethylene biosynthesis, defense gene expression, ion leakage, callose deposition, hypersensitive response (HR), PR protein activity, protease inhibitor activity.

The term “population” refers hereinafter to a group of organisms of a species that interbreed and live in same place at the same time. They are capable of interbreeding or reproduction (Jonathan Cumming, Biology Online. Retrieved 5 Dec. 2012).

The term “wild type (WT)” refers hereinafter to an organism, a phenotype, a genotype, or a gene that predominates in a natural population of organisms or strain of organisms in contrast to that of natural, laboratory, bioengineered mutant forms.

The term “single mutation” refers hereinafter to a mutation or an alteration in the nucleotide sequence of one gene.

The term “double mutant” refers hereinafter to an organism carrying two different mutated genes.

As used herein after, the term “about” refers to any value being up to 25% lower or greater the defined measure.

The term “increased defense response, “increasing defense response” refers hereinafter to an increase of about 10% of at least one defense response selected from the group consisting of: ethylene production, ion leakage, expression of at least one defense gene, HR, callose deposition, all calculated as further described, all in comparison to a wild type (WT) plant. Increased defense response can lead to a decrease of about 10% of relative disease levels or absolute lesion/necrosis/diseased area, calculated as further described.

The present invention provides a plant carrying two different mutated NRC4 (NRC4a and NRC4b) genes. Said mutant plant is a homozygous plant for both mutated NRC4a and NRC4b genes. Further said double mutant homozygous plant exhibits increased immune responses and disease resistance when compared with other single mutant plant lines or non-mutated plant lines of the same population. A plant line with an intrinsic increased defense response to pathogens is of a great importance to agriculture and the environment, advantageous to all parties of the agricultural chain from farmer to consumer.

Further the present invention provides methods for increasing plant pathogen induced defense response mutating both the NRC4a and the NRC4b immune defense related genes by means of the crispr/cas gene editing system.

In a preferred embodiment of the present invention, a method for increasing plant pathogen induced defense response to be used on a tomato plant expressing the NRC4a and NRC4b genes. This embodiment is by no means restricting and the method is further applicable to any plant expressing the NRC4a and NRC4b genes, NRC4a and NRC4b genes homologues or orthologues.

In yet another preferred embodiment of the present invention the gene editing mechanism deployed to obtain the homozygous double mutant plant is a crispricas9 system. The gene editing system and/or technique utilized to obtain the double mutant plant is not limited to the crispr/cas9 system and may be selected from any of the crisper/cas systems or meganucleases, zinc finger nucleases, TALEN, an unspecified gene editing agent and any combination thereof.

### Example 1

Generation of SlNRC4b and SlNRC4Ab Crispr and Double Mutants

For the present invention, tomato plants NRC4b gene was mutated using the gene editing system of crispr/cas. Two independent lines mutated in NRC4b (slnrc4b1-2 and slnrc4b5-2) were demonstrated to be bi-allelic homozygous mutants in SINRC4b. In addition, a single line mutated both in NRC4b and NRC4a (slnrc4ab9-1), to be bi-allelic homozygous mutants of SINRC4b and NRC4a.

According to Sequence analysis:
slnrc4b1-2 harbors a four-base deletion (nucleic acid position 178-181), resulting in a frame shift leading to two mutated aa followed by an early stop codon, resulting in a 61 aa (amino acid) truncated protein.
slnrc4b5-2 harbors a two-base deletion (nucleic acid position 178-179) of SlNRC4b, resulting in a frame shift leading to 11 mutated aa followed by an early stop codon, resulting in a 70 aa truncated protein.

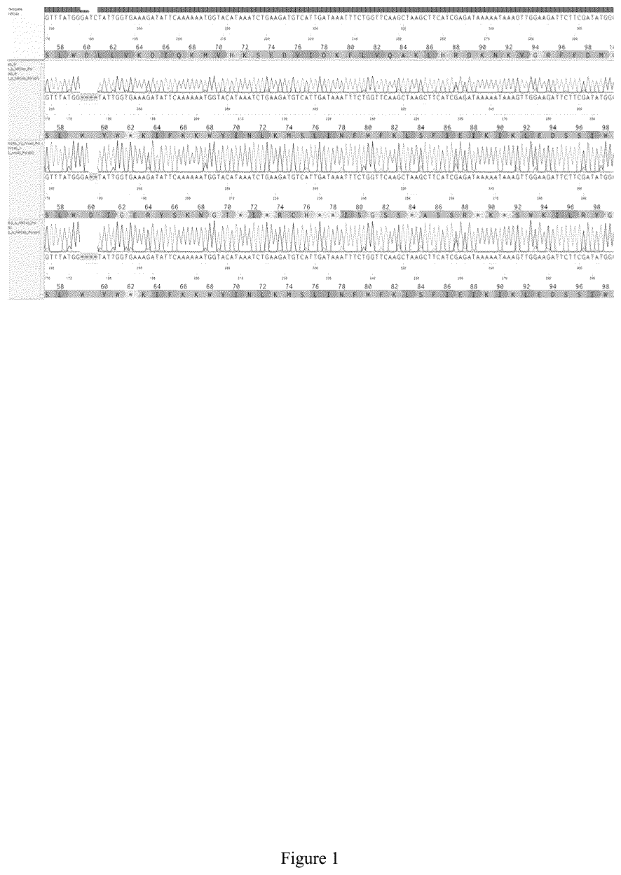

Reference in now made to FIG. 1 depicting the DNA and amino acid sequences of the NRC4b gene mutants: slnrc4b1-2, slnrc4b5-2 and slnrc4ab9-1.

slnrc4ab9-1 harbors the same mutation in NRC4b and express the same 62 aa protein as slnrc4b1-2, further it harbors a single base deletion of guanin, at position 170 nucleotides (57 aa position) of SINRC4a. The deletion resulting in a frame shift, producing 35 mutated aa followed by an early stop codon, resulting in a 91 aa truncated protein.

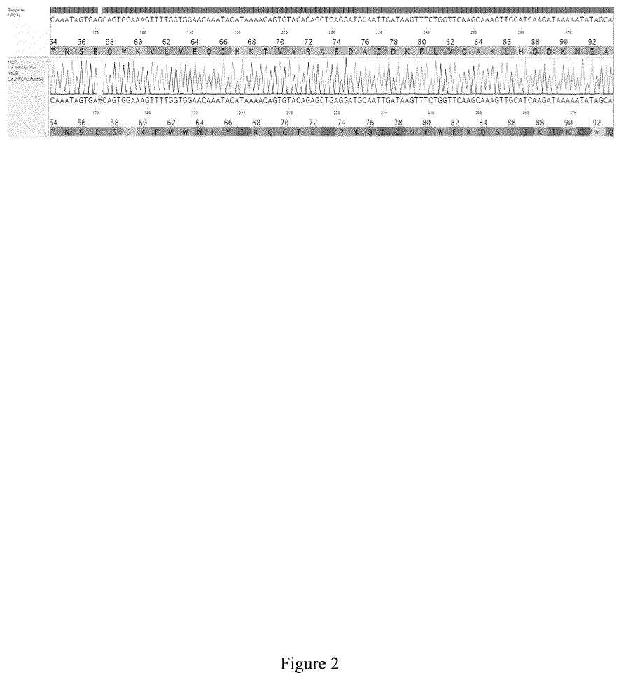

Reference in now made to FIG. 2 depicting the DNA and amino acid sequences of the mutated NRC4a gene of the double mutant slnrc4ab9-1.

### Example 2

Overexpression of the mutant NRC4a provides increased immune responses was previously reported (Leibman Markus et al., 2018). Assessing the immune responses of overexpression of the NRC4b in comparison to the previously reported mutant NRC4a, a very similar increase of reactive oxygen species (ROS) to the same levels as NRC4a.

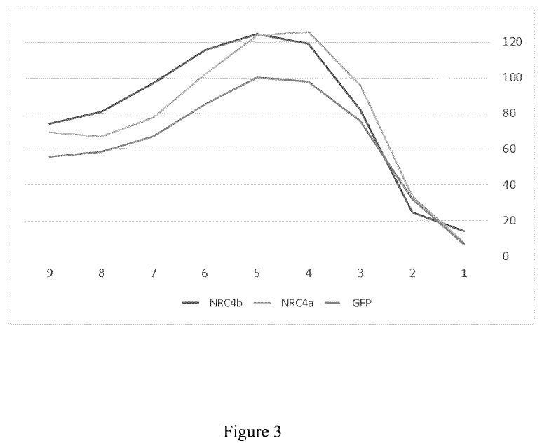

Reference in now made to FIG. 3 depicting the ROS levels of the overexpression of NRC4a, NRC4b and control.

### Example 3

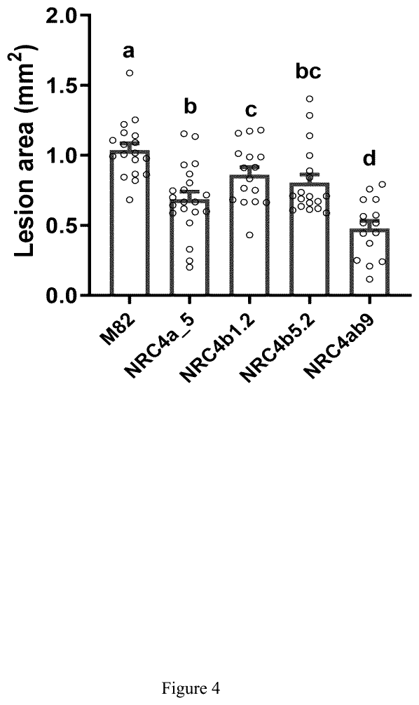

A NRC4a mutant was previously reported to have increased resistance to botrytis (Leibman-Markus et al 2018, Pizarro et al 2020). The present invention further discloses that each independent mutant display lower disease levels (lesion area) than the WT plants in response to B. cinerea infection. While NRC4a lines' diseases levels were lower than the two independent NRC4b, the double NRC4ab #9 line showed the lowest disease levels, hence the double NRC4ab #9 line is significantly more resistance than all NRC4 mutated lines of the present invention. In order to asses diseases levels, a pure B. cinerea (Bc16) culture was grown on potato dextrose agar (PDA) (Difco Lab) plates and incubated at 22° C. for 5-7 days. B. cinerea spores were harvested in 1 mg ml⁻¹ glucose and 1 mg ml⁻¹ K2HPO4 and filtered through cheesecloth. Spore concentration was adjusted to 10⁶ spores ml⁻¹ using a hemocytometer. Leaves 4-6 from 5-6 weeks old tomato plants were excised and immediately placed in humid chambers. Each tomato leaflet was inoculated with two droplets of 10 μL spores' suspension. Inoculated leaves were kept in humid growth chamber at 21° C. B. cinerea experiment was replicated four independent times, using leaves from five individuals for each genotype and 3-4 technical replicates. Reference in now made to FIG. 4 depicting disease levels measured in lesion area (mm) of the different genotypes, reinforcing that the double mutant NRC4ab #9 possess the highest disease resistance.

### Example 4

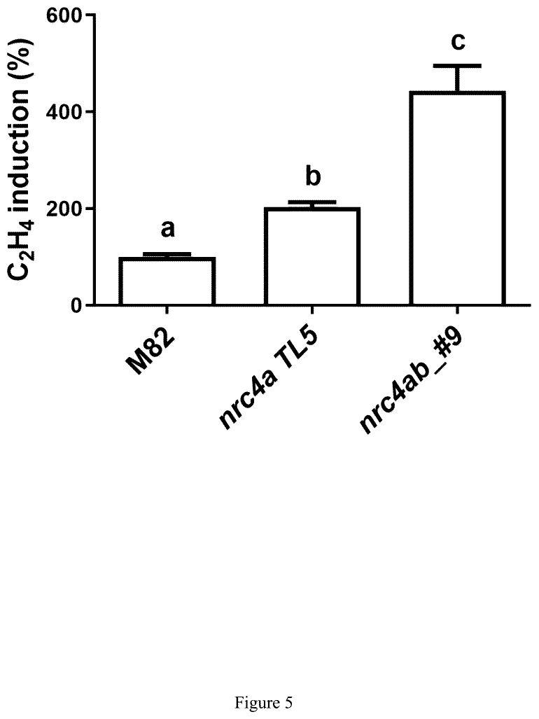

In response to xylanase, there is an increased ethylene production in the double NRC4ab #9 mutant, which is significantly higher than in all mutants even more than the NRC4a mutant alone, which was previously established to have higher ethylene production compared with the WT. Reference in now made to FIG. 5 depicting ethylene productions levels of all lines. Ethylene production was measured as previously described (Leibman-Markus et al., 2017). Leaf disks 0.9 cm in diameter were taken from leaves 4-6 of 5-6 weeks old M82 and slnrc4a, slnrc4b, or slnrc4ab tomato leaves. Disks were washed in water for 1-2 h. Every six disks were sealed in a 10 mL flask containing 1 ml assay medium (with or without 1 μg/mL EIX) for 4 h at room temperature. Ethylene production was measured by gas chromatography (Varian 3350, Varian, California, USA).

### Example 5

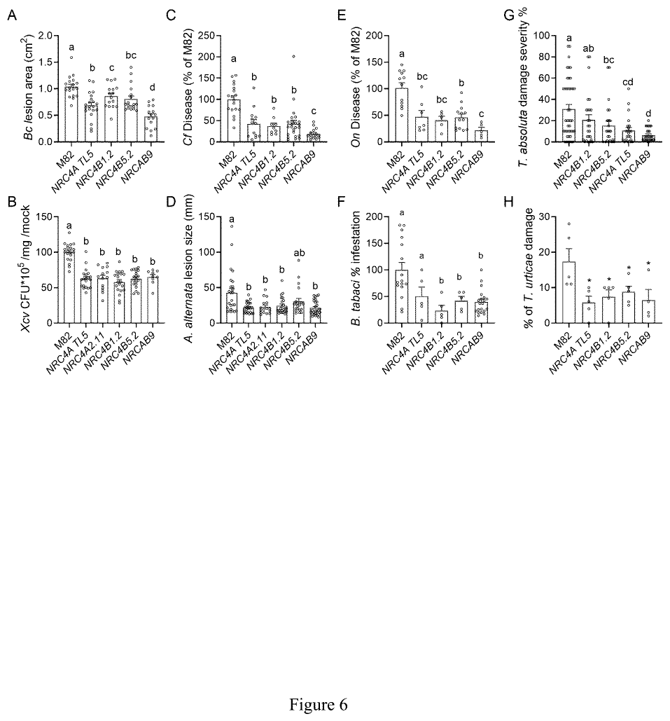

Moreover, the NRC mutants are more resistant to a variety of diseases, with the double mutant having significant advantages over the single mutants. Reference in now made to FIG. 6a-6h; FIG. 6a-6f depicting a graphic presentation of the increase resistance of the NRC4 mutants to various pathogens. Tomato plants of the indicated genotypes were challenged with B. cinerea (10⁶ conidia/mL) (FIG. 6a), X. euvesicatoria (10⁴ CFU/mL) (FIG. 6b), C. fulvum (10⁶ conidia/mL) (FIG. 6c), A. alternata (10⁶ conidia/mL) (FIG. 6d), O. neolycopersici (10⁴ conidia/mL) (FIG. 6e), or transferred to chambers infested with B. tabaci (FIG. 6f) or T absoluta (FIG. 6g-6h). Relative disease area was calculated as the lesion area measured 5 days after inoculation in each genotype for B. cinerea, and similarly, 10 days after inoculation, for O. neolycopersici, A. alternate, and C. fulvum. For X. euvesicatoria, pathogen levels were quantified 3 days after inoculation. For the pests B. tabaci (FIG. 6f), T absoluta (FIG. 6g), and T urticae (FIG. 6h), pest numbers and damage were evaluated 2 weeks after exposure. Bars represent average ±SEM, all points shown. Different letters represent statistically significant differences in a one-way ANOVA with a Tukey post hoc test. In all cases, experiments were repeated 3-6 independent times. (A) N>25, p<0.023 (B) N>9, p<0.0001 (C) N>9, p<0.03 (D) N>16, p<0.005 (E) N>5, p<0.042 (F) N>5, p<0.031 (G) N>23, p<0.04 (H) N>5, p<0.04. Therefore, it was demonstrated that NRC mutants are more resistant to a variety of diseases, with the double mutant having significant advantages over the single mutants (FIG. 6a, FIG. 6c, FIG. 6e and FIG. 6g), while retaining all the other agricultural qualities.

### Example 6

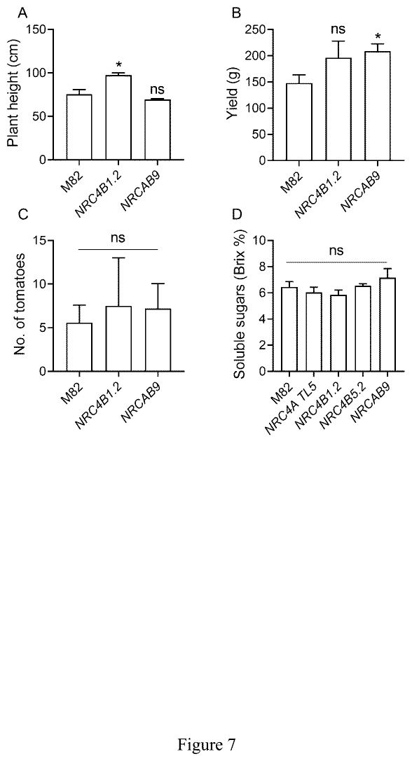

Furthermore, it was proved that NRC mutants are more resistant to a variety of diseases, since the double mutant also has increased yield per plant, while retaining all the other agricultural qualities. Reference in now made to FIG. 7a-7d. FIG. 7a-7d depicting a graphic representation of the phenotypic parameters of the NRC4 mutants compared to WT. After measuring the phenotypic parameters of yield in M82, nrc4a, nrc4b, and nrc4ab plants, it was also observed that no statistically significant differences were observed among genotypes in No. of tomatoes or Brix. NRC4ab has significantly increased yield production (FIG. 7b), in one-way ANOVA with Tukey's post hoc test (p<0.05). FIG. 7a represents the plant height; FIG. 7b the yield per plant in grams; FIG. 7c the number of tomato fruits produced by plant; and FIG. 7d the total soluble sugars were measured by refractometry expressed as ° Brix. Average ±SEM of 3-10 independent replicates is shown.

The invention is not intended to be limited to the embodiment illustrated and described above, but it can be modified and varied within the scope and spirit of the invention as defined by the following claims.

1.-72. (canceled)

73. A double mutant plant line harboring impaired NRC4a (NLR required for cell death) and NRC4b genes, said double mutant plant line characterized by at least one of the following:
an increased defense response compared to a non-mutated or a single mutation mutated plant of the same population; and
a reduction in disease levels compared to a non-mutated or a single mutation mutated plant of the same population.

74. The double mutant plant line according to claim 73, wherein said plant is a double mutant homozygous plant to the NRC4a and/or NRC4b impaired gene.

75. The double mutant plant line according to claim 73, wherein said plant expresses a truncated NRC4a and/or NRC4b proteins.

76. The double mutant plant line of claim 75, wherein at least one of said truncated proteins is characterized by at least one increased function compared to the full-length proteins.

77. The double mutant plant line according to claim 75, wherein at least one of said truncated proteins possess at least one enhanced activity of the native proteins.

78. The double mutant plant line according to claim 73, wherein said plant is a member of the Solanaceae family.

79. The double mutant plant line according to claim 73, wherein said plant is selected from the group consisting of Solanum lycopersicum, Nicotiana tabacum, Solanum tuberosum, Solanum melongena, Capsicum annum, Petunia hybrid, a NRC4a and/or NRC4b genes expressing plant, a NRC4a and/or NRC4b ortholog gene expressing plant, and any combination thereof.

80. The double mutant plant line according to claim 73, wherein said double mutant plant is mutated by means of a gene editing system.

81. The double mutant plant line of claim 80, wherein said gene editing system is selected from the group consisting of: gene editing agent, meganucleases, zinc finger nucleases, TALEN, Crispr/Cas and any combination thereof.

82. The double mutant plant line according to claim 80, wherein said gene editing system is a crispr/cas system.

83. The double mutant plant line according to claim 73, wherein said single mutation is located at either the NRC4a or NRC4b genes.

84. The double mutant plant line of claim 73, wherein said increased defense response or/and said reduction in disease levels is further increased upon interaction with a bio control agent (BCA).

85. The double mutant plant line according to claim 84, wherein said bio control agent is selected from the group consisting of:
insects;
microorganisms such as fungi, bacteria, oomycets, viruses; and
chemicals such as nucleic acids, hormones, hormone analogs, natural products, organic compounds.

86. A method for increasing a plant defense response, said method comprising the following steps:
configuring one or more nucleic acid sequences to target and impair at least two plant NRC (NLR required for cell death) or NRC ortholog genes;
applying a gene editing process utilizing said one or more nucleic acid sequences; and
obtaining at least one double mutant plant line, harboring two impaired NRC (NLR required for cell death) genes.

87. The method of claim 86, said method further comprising the step of growing said plant.

88. The method according of claim 86, wherein said plant is a double mutant homozygous to at least one of the NRC4a and NRC4b impaired gene.

89. A mutated plant line harboring an impaired NRC4b (NLR required for cell death) gene, said mutant plant line characterized by at least one of the following:
an increased defense response compared to a non-mutated plant of the same population; and
a reduction in disease levels compared to a non-mutated plant of the same population.

90. The mutated plant line according to claim 89, wherein said mutated plant line is homozygous to the impaired NRC4b gene.

91. The mutated plant line of claim 89, wherein said increased defense response or said reduction in disease levels is further increased upon interaction with a bio control agent (BCA).

92. The mutated plant line according to claim 91, wherein said bio control agent is selected from the group consisting of:
insects;
microorganisms such as fungi, bacteria, oomycets, viruses; and
chemicals such as nucleic acids, hormones, hormone analogs, natural products, organic compounds.
